(19) United States
(12) Patent Application Publication
(10) Pub. No.: US 2014/0148859 A1
(43) Pub. Date: May 29, 2014

(54) ORTHOPEDIC FUSION PLATE AND COMPRESSION SCREW

(71) Applicant: SOLANA SURGICAL, LLC, Memphis, TN (US)

(72) Inventors: Alan G. Taylor, Memphis, TN (US); Rebecca H. Wahl, Escondido, CA (US); Ronald George Litke, Jr., Sandy Hook, CT (US)

(73) Assignee: SOLANA SURGICAL, LLC, Memphis, TN (US)

(21) Appl. No.: 13/828,358

(22) Filed: Mar. 14, 2013

Related U.S. Application Data
(60) Provisional application No. 61/730,433, filed on Nov. 27, 2012.

(51) Int. Cl. A61B 17/80

(52) U.S. Cl. CPC A61B 17/8014
USPC 606/282

(57) ABSTRACT

A fusion plate (10) for compressing bone material at an interface between a first bone part (929A) and a second bone part (929B) comprises (i) a first end (12) having a first thickness (464); (ii) a thickened region (250) that is spaced apart from the first end (12), the thickened region (250) having a second thickness (466) that is greater than the first thickness (464); and (iii) a compression aperture (22) that extends through the thickened region (250) at a compression angle (224) relative to a planar axis (246). The compression angle (224) can be between approximately thirty and fifty-five degrees relative to the planar axis (246). The first end (12) includes a top surface (26), and the thickened region (250) extends farther away from the bone parts (929A, 929B) than the top surface (26) when the fusion plate (10) is implanted substantially adjacent to the bone parts (929A, 929B).

### RELATED INVENTION

This application claims priority on U.S. Provisional Application Ser. No. 61/730,433, filed Nov. 27, 2012 and entitled “ORTHOPEDIC FUSION PLATE AND COMPRESSION SCREW”. As far as permitted, the contents of U.S. Provisional Application Ser. No. 61/730,433 are incorporated herein by reference.

### BACKGROUND

An orthopedic fusion plate (also referred to herein simply as a “fusion plate”) can be utilized in conjunction with a compression screw (or other suitable “compression attacher”) to increase compression at a bone interface. Moreover, a fusion plate with a compression attacher can function to stabilize bones or bone parts relative to each other so as to cause fusion. In certain applications, fusion plates and/or compression attachers can be used to fuse bones and/or bones parts in the foot, the ankle, the hand, and/or the wrist of the human body. Additionally and/or alternatively, fusion plates and/or compression attachers can be used in different portions of the body to stabilize and/or fuse bones or bone parts relative to each other.

### SUMMARY

The present invention is directed toward a fusion plate for compressing bone material at an interface between a first bone part and a second bone part, the fusion plate including a planar axis that extends along a length of the fusion plate. In certain embodiments, the fusion plate comprises (i) a first end having a first thickness; (ii) a thickened region that is spaced apart from the first end, the thickened region having a second thickness that is greater than the first thickness; and (iii) a compression aperture that extends through the thickened region at a compression angle relative to the planar axis. In some such embodiments, the compression angle is between approximately thirty and fifty-five degrees relative to the planar axis.

Additionally, in some embodiments, the first end includes a top surface, and the thickened region extends in a generally upward direction away from the top surface, such that the thickened region extends farther away from the bone parts than the top surface when the fusion plate is implanted substantially adjacent to the bone parts. In one such embodiment, the second thickness is at least approximately 1.0 millimeters greater than the first thickness.

Further, in one embodiment, the fusion plate further comprises one or more plate lifters that are positioned along and extend away from a bottom surface of the fusion plate, the one or more plate lifters being adapted to engage at least one of the bone parts when the fusion plate is implanted substantially adjacent to the bone parts.

In certain embodiments, the fusion plate further comprises a first fixation aperture that extends through the fusion plate near the first end. In one such embodiment, the fusion plate further comprises a second end that is spaced apart from the first end, and a second fixation aperture that extends through the fusion plate near the second end. Additionally, in such embodiment, the thickened region is positioned within a middle region substantially between the first end and the second end.

The present invention is further directed toward a combination comprising the fusion plate as described above, and a compression attacher, e.g., a compression screw, that extends through the compression aperture, the compression attacher being adapted to extend into the first bone part and the second bone part when the fusion plate is implanted substantially adjacent to the bone parts. In one embodiment, the compression attacher includes (i) a threaded region including a plurality of threads having a threaded width, and (ii) an attacher width, and wherein the threaded width is greater than the attacher width.

Additionally, in one application, the present invention is directed toward a fusion plate for compressing bone material at an interface between a first bone part and a second bone part, the fusion plate including a planar axis that extends along a length of the fusion plate, the fusion plate comprising (i) a top surface; (ii) an opposed bottom surface; and (iii) one or more plate lifters that are positioned along and extend away from the bottom surface of the fusion plate, the one or more plate lifters adapted to engage at least one of the bone parts when the fusion plate is implanted substantially adjacent to the bone parts, the one or more plate lifters having a buttress design. In such application, the fusion plate can further comprise a compression aperture that extends through the top surface and the bottom surface at a compression angle relative to the planar axis.

Further, in one application, the present invention is further directed toward a combination for compressing bone material at an interface between a first bone part and a second bone part, combination comprising (A) a fusion plate including (i) a first end having a top surface and a first thickness, (ii) an opposed second end that is spaced apart from the first end, (iii) a thickened region that is positioned within a middle region substantially between the first end and the second end, the thickened region extending in a generally upward direction away from the top surface, such that the thickened region extends farther away from the bone parts than the top surface when the combination is implanted substantially adjacent to the bone parts, the thickened region having a second thickness that at least approximately 1.0 millimeters greater than the first thickness, and (iv) a compression aperture that extends through the thickened region at a compression angle of between approximately thirty and fifty-five degrees relative to the planar axis; and (B) a compression screw that extends through the compression aperture, the compression screw being adapted to extend into the first bone part and the second bone part when the combination is implanted substantially adjacent to the bone parts.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a top view of an embodiment of a fusion plate having features of the present invention;

FIG. 2 is a sectional view of the fusion plate taken on line X-X in FIG. 1;

FIG. 3 is an end view of the fusion plate illustrated in FIG. 1, and a plurality of fixation attachers and a compression attacher that can be used with the fusion plate;

FIG. 4 is a sectional view of the fusion plate and the plurality of fixation attachers and the compression attacher taken on line A-A in FIG. 3;

FIG. 5 is a bottom view of the fusion plate and the plurality of fixation attachers and the compression attacher illustrated in FIG. 3;

FIG. 6 is an enlarged view of a portion of the fusion plate as indicated by circle and arrow A-A in FIG. 4;

FIG. 7 is an enlarged view of a portion of the fusion plate as indicated by circle and arrow B-B in FIG. 4;

FIG. 8A is a side view of an embodiment of a compression attacher having features of the present invention;

FIG. 8B is a sectional view of the compression attacher taken on line B-B in FIG. 8A;

FIG. 8C is a top end view of the compression attacher illustrated in FIG. 8A;

FIG. 8D is a bottom end view of the compression attacher illustrated in FIG. 8A;

FIG. 8E is an enlarged view of a portion of the compression attacher as indicated by circle and arrow E-E in FIG. 8B;

FIG. 8F is an enlarged view of a portion of the compression attacher as indicated by circle and arrow F-F in FIG. 8B;

FIG. 9 is a simplified schematic illustration of a first bone part, a second bone part, and an embodiment of the fusion plate, the fixation attachers and the compression attacher having features of the present invention that are utilized to hold the bone parts together;

FIG. 10A is a top view of another embodiment of a fusion plate having features of the present invention;

FIG. 10B is a sectional view of the fusion plate taken on line B-B in FIG. 10A;

FIG. 11A is a top view of still another embodiment of a fusion plate having features of the present invention;

FIG. 11B is a sectional view of the fusion plate taken on line B-B in FIG. 11A;

FIG. 11C is a sectional view of the fusion plate taken on line B-B in FIG. 11A, and another embodiment of a compression attacher;

FIG. 12A is a top view of yet another embodiment of a fusion plate having features of the present invention;

FIG. 12B is a sectional view of the fusion plate taken on line B-B in FIG. 12A;

FIG. 13A is a top view of still another embodiment of a fusion plate having features of the present invention;

FIG. 13B is a sectional view of the fusion plate taken on line B-B in FIG. 13A;

FIG. 14A is a top view of still yet another embodiment of a fusion plate having features of the present invention, and a plurality of fixation attachers and a compression attacher that can be used with the fusion plate; and

FIG. 14B is a sectional view of the fusion plate, the fixation attachers and the compression attacher taken on line B-B in FIG. 14A.

### DESCRIPTION

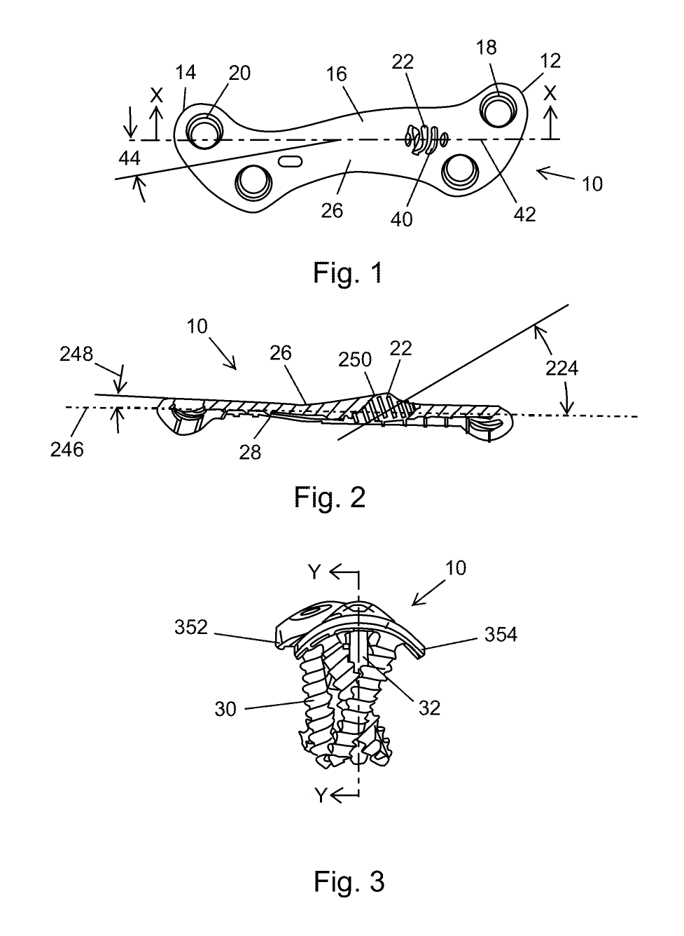

FIG. 1 is a top view of an embodiment of a fusion plate 10 having features of the present invention. The design of the fusion plate 10 can be varied. As shown in FIG. 1, the fusion plate 10 can include a first end 12, an opposed second end 14, and a middle region 16 that extends between and connects the first end 12 and the second end 14. Additionally, in this embodiment, the fusion plate 10 includes (i) one or more first fixation apertures 18 positioned near the first end 12 of the fusion plate 10 (two are illustrated in FIG. 1) with any appropriate positioning relative to one another, wherein each first fixation aperture 18 may extend at a slight angle completely through the fusion plate 10; (ii) one or more second fixation apertures 20 positioned near the second end 14 of the fusion plate 10 (two are illustrated in FIG. 1) with any appropriate positioning relative to one another, wherein each second fixation aperture 20 may extend at a slight angle completely through the fusion plate 10; and (iii) a compression aperture 22 positioned within the middle region 16 of the fusion plate 10, the compression aperture 22 extending at a compression angle 224 (illustrated in FIG. 2) completely through the fusion plate 10.

It should be noted that the use of the terms “first” and “second” to describe the ends and fixation apertures of the fusion plate 10 is merely for purposes of illustration and ease of description and is not intended to be limiting in any manner. Further, either end and/or either fixation apertures can equally be titled as “first” or “second” without altering the breadth and scope of the present invention

Additionally, it should be noted that as utilized herein, the concept of the apertures 18, 20, 22 extending completely through the fusion plate 10 signifies that the apertures 18, 20, 22 extend through both a top surface 26 and a bottom surface 28 (illustrated most clearly in FIG. 5) of the fusion plate 10. Further, as utilized herein, the top surface 26 is the surface of the fusion plate 10 that faces away from the bones or bone parts, e.g., a first bone part 929A and a second bone part 929B (illustrated in FIG. 9), when the fusion plate 10 is implanted, and the bottom surface 28 is the surface of the fusion plate 10 that faces toward the bone parts 929A, 929B when the fusion plate 10 is implanted.

In certain embodiments, each of the apertures 18, 20, 22 can be internally threaded. Alternatively, the apertures 18, 20, 22 can be designed such that one or more of the apertures 18, 20, 22 are not internally threaded.

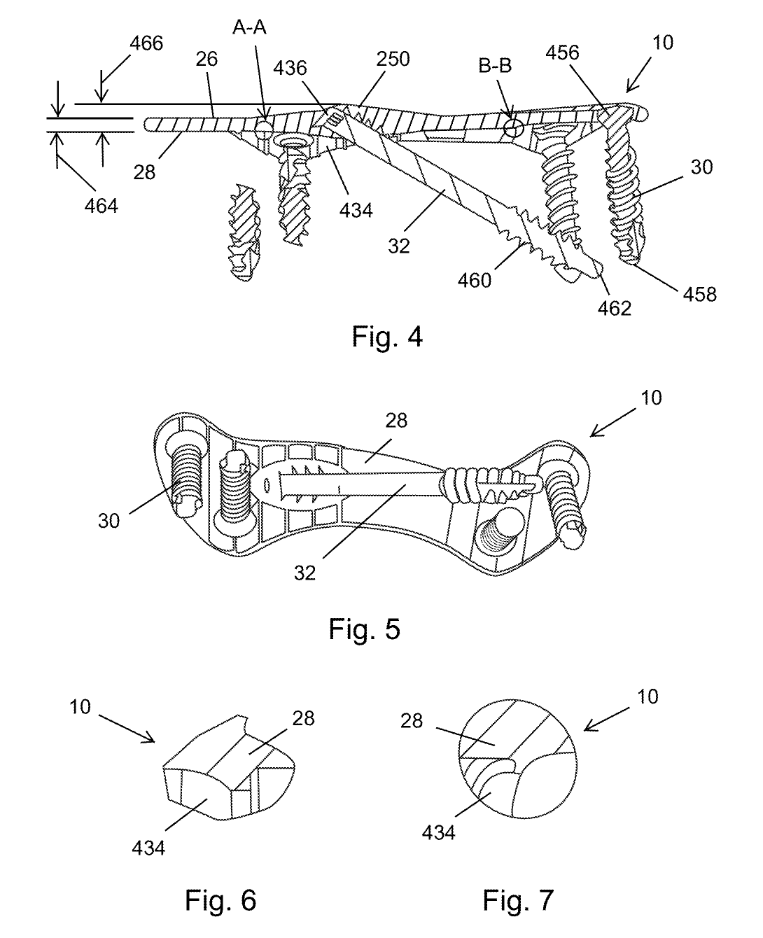

Additionally, each of the fixation apertures 18, 20 is adapted to receive a fixation attacher 30 (as illustrated, for example, in FIG. 3), e.g., a screw, and the compression aperture 22 is adapted to receive a compression attacher 32 (as illustrated, for example, in FIG. 4), e.g., a compression screw.

Further, the number of first fixation apertures 18 and the number of second fixation apertures 20 can be varied. For example, as illustrated in FIG. 1, the fusion plate 10 can include two first fixation apertures 18 and two first fixation apertures 20. Alternatively, the fusion plate 10 can include greater than two or less than two first fixation apertures 18 (e.g., zero, one or three first fixation apertures 18); and/or the fusion plate 10 can include greater than two or less than two second fixation apertures 20 (e.g., zero, one or three second fixation apertures 20).

As an overview, the fusion plate 10 is designed to be fixed relative to the first bone part 929A and the second bone part 929B via the attachers 30, 32 that are threaded through and/or extend through the apertures 18, 20, 22 in the fusion plate 10. For example, (i) the fixation attachers 30 that are threaded through and/or extend through the first fixation apertures 18 can be threaded into the first bone part 929A; (ii) the fixation attachers 30 that are threaded through and/or extend through the second fixation apertures 20 can be threaded into the second bone part 929B; and (iii) the compression attacher 32 that is threaded through and/or extends through the compression aperture 22 can be threaded into both the first bone part 929A and the second bone part 929B. Additionally, the fusion plate 10 is designed to enable the effective compression and/or to increase the compression of bone material at a bone interface between the bone parts 929A, 929B. This compression is accomplished by extending the compression attacher 32, e.g., the compression screw, through the compression aperture 22 at the compression angle 224 and into the bone parts, i.e. into both the first bone part 929A and the second bone part 929B, to compress the bone parts 929A, 929B together.

Further, the fusion plate 10 is designed to provide increased stability in the bone parts 929A, 929B relative to one another such as to cause fusion. Importantly, the fusion plate 10 is able to accomplish these goals without the need for removal of any bone material to allow for the proper implantation, installment or insertion of the fusion plate 10. Moreover, as detailed herein below, the fusion plate 10 further includes one or more plate lifters 434 (illustrated in FIG. 4) along the bottom surface 28 of the fusion plate 10, which have a unique buttress design for relieving compression along the surfaces of the bone parts 929A, 929B, but which engage into the bone parts 929A, 929B and help fix the bone parts 929A, 929B in place for successful fusion.

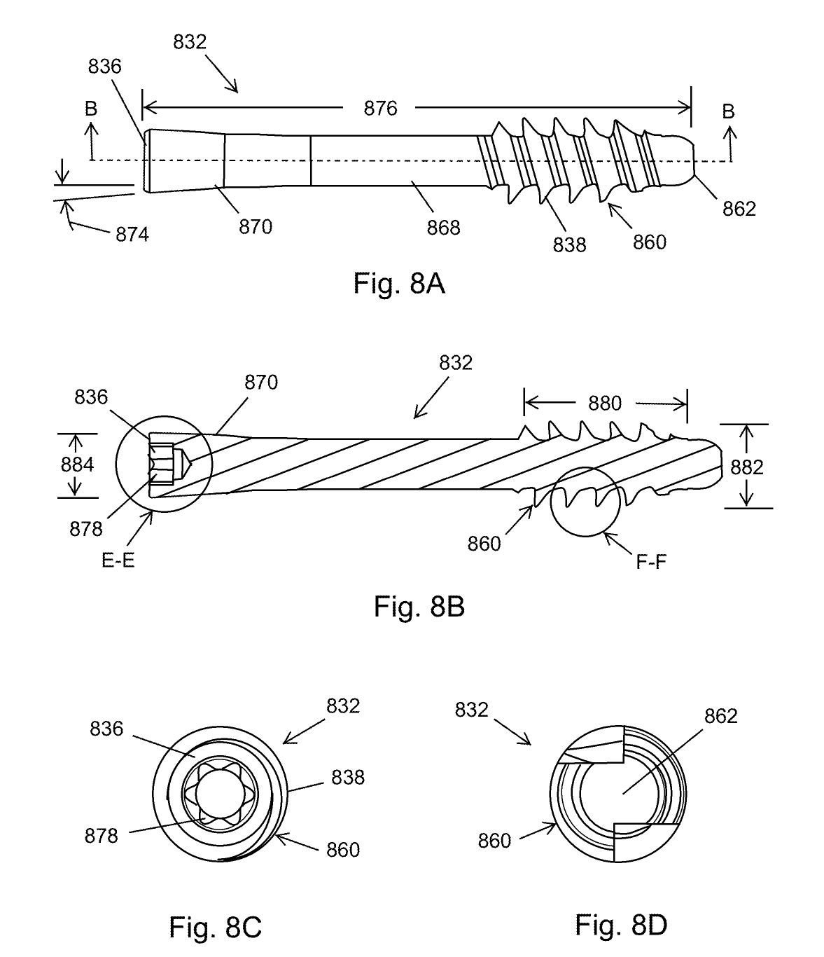

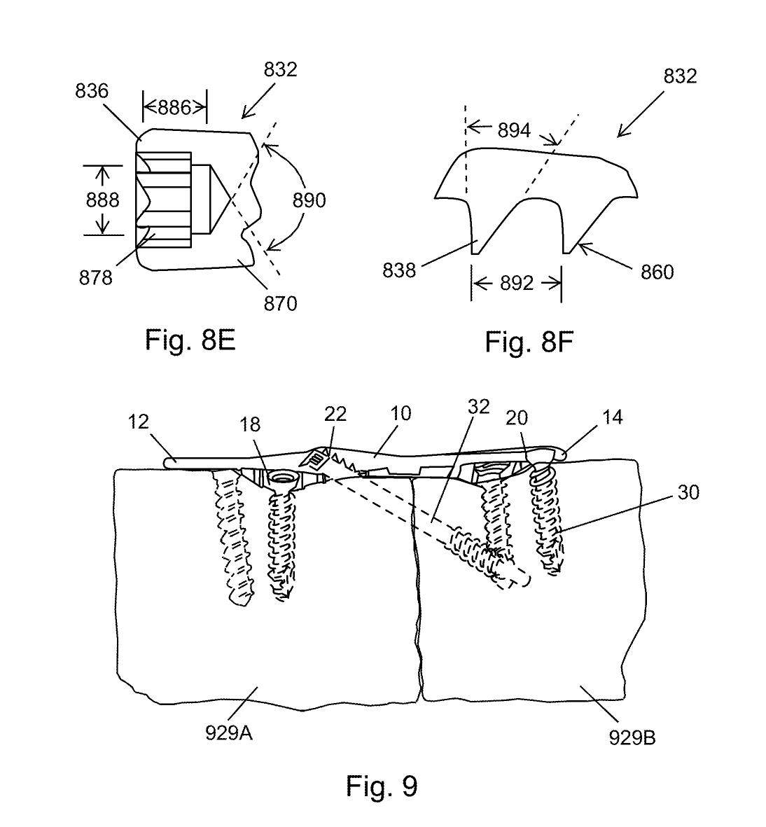

Moreover, as detailed herein below, the compression attacher 32 can be uniquely designed to allow for a low profile attacher-to-plate interface. For example, the compression attacher 32 can include a unique conical shape and/or a tapered head 836 (illustrated in FIG. 8A) so as to enable such a low profile attacher-to-plate interface. Further, in certain embodiments, the compression attacher 32 can have threads 838 (illustrated in FIG. 8A) that are larger than the head 836. In such embodiments, threads 40 can be formed into the compression aperture 22 to accommodate the size of the threads 838 of the compression attacher 32, and the smaller head 836 can be allowed to pass completely through the compression aperture 22 when and if desired, e.g., if the fusion plate 10 is to be removed, but it is desired to maintain the compression attacher 32 implanted within the bone parts 929A, 929B.

During proper implantation, installment or insertion of the fusion plate 10, typically one or more fixation attachers 30 are initially extended, e.g., threaded, through the first fixation apertures 18 at the first end 12 or the second fixation apertures 20 at the second end 14, and attached to one of the bone parts 929A, 929B. Subsequently, the compression attacher 32 is extended, e.g., threaded, through the compression aperture 22 and from the first bone part 929A to the second bone part 929B, to compress and fuse the bone parts 929A, 929B together. Then, the remaining fixation apertures 30 are extended through the remaining fixation apertures 18, 20 and into the respective bone parts 929A, 929B so that the fusion plate 10 is fixedly secured in place.

Further, as shown in FIG. 1, the fusion plate 10 can have a longitudinal axis 42, and a portion of the fusion plate 10, e.g., the second end 14 of the fusion plate 10, can further be angled at a valgus angle 44 relative to the longitudinal axis 42 to compensate for any valgus deformities that may exist in the bone parts 929A, 929B into and/or adjacent to which the fusion plate 10 is implanted. For example, in certain embodiments, the fusion plate 10 can have a valgus angle 44 of between approximately zero and fifteen degrees relative to the longitudinal axis 42. More specifically, in certain non-exclusive alternative embodiments, the fusion plate 10 can have a valgus angle 44 of approximately two, four, six, eight, ten, twelve or fourteen degrees relative to the longitudinal axis 42. Alternatively, the fusion plate 10 can have a valgus angle 44 relative to the longitudinal axis 42 that is different than the dimensions specifically mentioned above. For example, the fusion plate 10 can have a valgus angle 44 of zero degrees (i.e. no valgus angle), greater than fifteen degrees, or some other value between zero and fifteen degrees, relative to the longitudinal axis 42.

Moreover, as illustrated, in certain embodiments, the compression aperture 22 can extend substantially along and/or parallel to the longitudinal axis 42. Alternatively, the compression aperture 22 can be formed into the fusion plate 10 such that the compression aperture 22 extends at an angle relative to the longitudinal axis 42.

Additionally, in various embodiments, the fusion plate 10 can be made from titanium, stainless steel, polyether ether ketone (PEEK), a cobalt-chromium alloy, or another suitable material.

FIG. 2 is a sectional view of the fusion plate 10 taken on line X-X in FIG. 1. As illustrated in FIG. 2, the fusion plate 10 has a planar axis 246 that extends along a length of the fusion plate 10. Additionally, the fusion plate 10 includes a flexion angle 248 for dorsal flexion (or dorsiflexion) that is measured relative to the planar axis 246. For example, in certain embodiments, the fusion plate 10 can have a flexion angle 248 of between approximately zero and ten degrees in either direction relative to the planar axis 246. Stated in another manner, the second end 14 can flex and/or extend generally upward or downward from the planar axis 246 between approximately zero and ten degrees relative to the remainder of the fusion plate 10. More specifically, in some non-exclusive alternative embodiments, the fusion plate 10 can have a flexion angle 248 of approximately zero, one, two, three, four, five, six, seven, eight, nine or ten degrees relative to the planar axis 246. Alternatively, the fusion plate 10 can have a flexion angle 248 relative to the planar axis 246 that is different than the dimensions specifically mentioned above. For example, the fusion plate 10 can have a flexion angle 248 of greater than ten degrees, or some other value between zero and ten degrees relative to the planar axis 246.

Moreover, as shown in FIG. 2, the compression aperture 22 extends completely through the fusion plate 10 at a compression angle 224 relative to the planar axis 246. For example, in certain embodiments, the compression aperture 22 can extend completely through the fusion plate 10 at a compression angle 224 of between approximately 30.0 and 55.0 degrees relative to the planar axis 246. More specifically, in certain non-exclusive alternative embodiments, the compression aperture 22 can extend completely through the fusion plate 10 at a compression angle 224 of approximately 30.0, 32.5, 35.0, 37.5, 40.0, 42.5, 45.0, 47.5, 50.0, 52.5 or 55.0 degrees relative to the planar axis 246. Alternatively, the compression aperture 22 can extend completely through the fusion plate 10 at a compression angle 224 that is different than the dimensions specifically mentioned above. For example, the compression aperture 22 can extend completely through the fusion plate 10 at a compression angle 224 of greater than 55.0 degrees, less than 30.0 degrees, or some other value between 30.0 and 55.0 degrees relative to the planar axis 246.

Further, as shown in the embodiment illustrated in FIG. 2, the fusion plate 10 can include a thickened region 250 within the middle region 16 of the fusion plate 10, and the compression aperture 22 can extend through the thickened region 250 of the fusion plate 10. As illustrated, the thickened region 250 can be formed into the fusion plate 10 such that the thickened region 250 extends in a generally upward direction away from the top surface 26, i.e. extends farther away from the bone parts 929A, 929B when the fusion plate 10 is implanted. Stated in another manner, the additional thickness provided by the thickened region 250 is at least primarily, if not entirely, added onto the top surface 26 and extends upwardly away from the top surface 26 of the remainder of the fusion plate 10. Additionally and/or alternatively, at least a portion of the additional thickness provided by the thickened region 250 can extend away from the bottom surface 28 of the fusion plate 10.

The thickness of the thickened region 250 relative to the remainder of the fusion plate 10 can be varied depending on the size of the head, e.g., the head 836 (illustrated in FIG. 8A) or head 436 (illustrated in FIG. 4), of the compression attacher 32. In certain embodiments, the thickened region 250 can be between approximately 0.5 millimeters (approximately 0.02 inches) and 1.5 millimeters (approximately 0.06 inches) thicker than the remainder of the fusion plate 10. For example, in certain non-exclusive alternative embodiments, the thickened region 250 is at least approximately 0.5, 0.6, 0.7, 0.8, 0.9, 1.0, 1.1, 1.2, 1.3, 1.4 or 1.5 millimeters thicker than the remainder of the fusion plate 10. Alternatively, the thickened region 250 can be greater than 1.5 millimeters, less than 0.5 millimeters, or some other value between 0.5 and 1.5 millimeters thicker than the remainder of the fusion plate 10.

So as not to create a soft tissue irritation, with this design, the compression attacher 32 (illustrated in FIG. 4) can be positioned within and extend through the compression aperture 22 with no portion of the head, e.g., the head 836, or the head 436, of the compression attacher 32 extending above the top surface 26 of the fusion plate 10 or below the bottom surface 28 of the fusion plate 10. Stated in another fashion, the additional thickness of the thickened region 250 and the positioning of the compression aperture 22 through the thickened region 250 of the fusion plate 10 enables the entire head 436, 836 of the compression attacher 32 to be flush with the top surface 26 or effectively hidden within the fusion plate 10 during use. Accordingly, the fusion plate 10 can be implanted, installed or inserted into the patient, with the compression attacher 32 extending through the compression aperture 22 at the compression angle 224, without any unnecessary discomfort for the patient, and without the need to remove any bone material from the bone parts 929A, 929B (illustrated in FIG. 9) which are being compressed and/or fused together by the fusion plate 10.

FIG. 3 is an end view of the fusion plate 10 illustrated in FIG. 1, and a plurality of attachers, i.e. the fixation attachers 30 and the compression attacher 32, that can be used with the fusion plate 10. In this embodiment, each of the fixation attachers 30 can be threaded and/or extend through one of the first fixation apertures 18 (illustrated in FIG. 1) or through one of the second fixation apertures 20 (illustrated in FIG. 1), and the compression attacher 32 can be threaded or extend through the compression aperture 22 (illustrated in FIG. 1).

Additionally, FIG. 3 further illustrates that the fusion plate 10 can be curved or arched from a first side 352 to a second side 354 to enable a more snug fit to the bone parts 929A, 929B (illustrated in FIG. 9), which are being compressed and/or fused together by the fusion plate 10. The degree or extent of curvature of the fusion plate 10 from the first side 352 to the second side 354 can be varied as necessary to suit the specific design requirements of the fusion plate 10.

FIG. 4 is a sectional view of the fusion plate 10 and the attachers 30, 32 taken on line A-A in FIG. 3. More particularly, FIG. 4 illustrates a longitudinal sectional view of the fusion plate 10 and the attachers 30, 32 that cuts fully through the compression attacher 32.

The design of the fixation attachers 30 can be varied. In the embodiment shown in FIG. 4, each of the fixation attachers 30 has a design that is substantially similar to the design of each of the other fixation attachers 30. For example, as illustrated, each of the fixation attachers 30 is substantially fully threaded from a head 456 to a tip 458 of the fixation attacher 30. Alternatively, one or more of the fixation attachers 30 can have a design that is different from the other fixation attachers 30, e.g., one or more of the fixation attachers 30 can include less threading as compared to the fixation attachers 30 specifically illustrated in FIG. 4.

Additionally, the design of the compression attacher 32 can also be varied. In the embodiment shown in FIG. 4, the compression attacher 32 includes only a relatively small, threaded region 460 near a tip 462 of the compression attacher 32, with no threading being located near the head 436 of the compression attacher 32. Alternatively, the compression attacher 32 can include more threading or less threading (e.g., no threading) as compared to the compression attacher 32 specifically illustrated in FIG. 4.

Further, the specific design of the head 436 can also be varied. For example, as shown in FIG. 4, the head 436 of the compression attacher 32 can be substantially flat, e.g., the compression attacher 32 can be a flat-head screw. Alternatively, the head 436 of the compression attacher 32 can have a different design. For example, in certain non-exclusive alternative embodiments, the head 436 of the compression attacher 32 can be rounded and/or the head 436 can be tapered.

Moreover, as shown in FIG. 4 and as noted above, when the compression attacher 32 is positioned within the compression aperture 22 (illustrated more clearly in FIG. 1), the head 436 of the compression attacher 32 does not extend above the top surface 26 or below the bottom surface 28 of the fusion plate 10 due to the compression aperture 22 extending through the thickened region 250 of the fusion plate 10.

Additionally, as illustrated, the majority of the fusion plate 10 has a plate thickness 464 and the thickened region 250 has a maximum region thickness 466 that is greater than the plate thickness 464. In certain embodiments, the majority of the fusion plate 10 can have a plate thickness 464 of between approximately 0.03 and 0.06 inches, and the thickened region 250 can have a maximum region thickness 466 of between approximately 0.08 and 0.13 inches. For example, in certain non-exclusive alternative embodiments, the majority of the fusion plate 10 has a plate thickness 464 of approximately 0.03, 0.035, 0.04, 0.045, 0.05, 0.055 or 0.06 inches, and the thickened region 250 has a maximum region thickness 466 of approximately 0.08, 0.085, 0.09, 0.095, 0.10, 0.105, 0.11, 0.115, 0.12, 0.125 or 0.13 inches. In one specific non-exclusive example, the majority of the fusion plate 10 has a plate thickness 464 of approximately 0.047 inches, and the thickened region 250 has a maximum region thickness 466 of approximately 0.105 inches. As such, the difference between the plate thickness 464 and the maximum region thickness 466 is approximately 0.058 inches. Thus, in such embodiment, the maximum region thickness 466 is approximately one hundred twenty-three percent greater than the plate thickness 464. Alternatively, the plate thickness 464 can be greater than 0.06 inches, less than 0.03 inches, or some other value between 0.03 and 0.06 inches; and/or the maximum region thickness 466 can be greater than 0.13 inches, less than 0.08 inches, or some other value between 0.08 and 0.13 inches.

Further, FIG. 4 also illustrates the one or more plate lifters 434 along the bottom surface 28 of the fusion plate 10. As illustrated, the plate lifters 434 have a unique buttress design, which enable the plate lifters 434 to relieve compression along the bone surfaces of the bone parts 929A, 929B (illustrated in FIG. 9), but also to engage into the bone parts 929A, 929B and help fix the bone parts 929A, 929B in place for successful fusion.

FIG. 5 is a bottom view of the fusion plate 10 and the attachers 30, 32 illustrated in FIG. 3. In particular, FIG. 5 illustrates the attachers 30, 32 extending through and away from the bottom surface 28 of the fusion plate 10. Additionally, FIG. 5 further illustrates how each of the fixation attachers 30 can extend through and away from the bottom surface 28 at an angle that is different than each of the other fixation attachers 30. In one embodiment, the angles at which each of the individual fixation attachers 30 extend through and away from the bottom surface 28 can relate to the curvature or arching of the fusion plate 10 from the first side 352 to the second side 354 as shown in FIG. 3.

FIG. 6 is an enlarged view of a portion of the fusion plate 10 as indicated by circle and arrow A-A in FIG. 4. In particular, FIG. 6 illustrates an enlarged view of a portion of the connection between the bottom surface 28 of the fusion plate 10 and the plate lifters 434.

FIG. 7 is an enlarged view of a portion of the fusion plate 10 as indicated by circle and arrow B-B in FIG. 4. In particular, FIG. 7 illustrates an enlarged view of another portion of the connection between the bottom surface 28 of the fusion plate 10 and the plate lifters 434.

FIG. 8A is a side view of an embodiment of a compression attacher 832 having features of the present invention. The design of the compression attacher 832 can be varied to suit the specific requirements of the fusion plate 10 (illustrated in FIG. 1) and/or the specific procedure being performed. In certain embodiments, the compression attacher 832 is uniquely designed to enable a low profile attacher-to-plate interface. In the embodiment illustrated in FIG. 8A, the compression attacher 832 includes a threaded region 860, a shaft 868, a tapered region 870, and a head 836.

The threaded region 860 is located near or substantially adjacent to a tip 862 of the compression attacher 832. The threaded region 860 is threaded, i.e. includes a plurality of threads 838, to enable the compression attacher 832 to be screwed into and thus retained within the bone parts 929A, 929B (illustrated in FIG. 9). The length of the threaded region 860, the number and size of the threads 838, and/or the pitch of the threads 838 can be varied depending on the specific requirements of the compression attacher 832.

The shaft 868 extends between the threaded region 860 and the tapered region 870 of the compression attacher 832. In certain embodiments, the shaft 868 can be substantially cylinder shaped to enable the shaft 868 to fit comfortably within the bone parts 929A, 929B upon insertion of the compression attacher 832. Alternatively, the shaft 868 can have another suitable shape.

The tapered region 870 extends between the shaft 868 and the head 836 of the compression attacher 832. In certain embodiments, the tapered region 870 can angle outward from the shaft 868 at a taper angle 874 of between approximately 2.0 and 8.0 degrees. For example, in certain non-exclusive alternative embodiments, the tapered region 870 can angle outward from the shaft 868 at a taper angle 874 of approximately 2.0, 2.5, 3.0, 3.5, 4.0, 4.5, 5.0, 5.5, 6.0, 6.5, 7.0, 7.5 or 8.0 degrees. Alternatively, the tapered region 854 can angle outward from the shaft 852 at a tape angle 874 of greater than 8.0 degrees, less than 2.0 degrees, or some other value between 2.0 and 8.0 degrees.

The head 836 of the compression attacher 832 is designed to be engaged by an insertion tool (not illustrated). Additionally, the head 836 is designed to have a relatively low profile to enable a low profile attacher-to-plate interface. For example, in certain embodiments, the head 836 of the compression attacher 32 can have threads 872 that are larger (i.e. wider in circumference) than the head 836. In such embodiments, the smaller head 836 can be allowed to pass completely through the compression aperture 22 (illustrated in FIG. 1) when and if desired, e.g., if the fusion plate 10 is to be removed, but it is desired to maintain the compression attacher 832 implanted within the bone parts 929A, 929B.

In certain embodiments, the compression attacher 832 can have an overall length 876 of between approximately 0.7 inches and 1.6 inches depending on the specific requirements of the bone parts 929A, 929B into which the fusion plate 10 (illustrated in FIG. 1) is being inserted. For example, in certain non-exclusive alternative embodiments, the length 876 of the compression attacher 832 can be approximately 0.7, 0.8, 0.9, 1.0, 1.1, 1.2, 1.3, 1.4, 1.5 or 1.6 inches. Alternatively, the length 876 of the compression attacher 832 can be greater than 1.6 inches, less than 0.7 inches, or some other value between 0.7 and 1.6 inches.

Additionally, in various embodiments, the compression attacher 832 can be made from titanium, stainless steel, polyether ether ketone (PEEK), a cobalt-chromium alloy, or another suitable material.

FIG. 8B is a sectional view of the compression attacher 832 taken on line B-B in FIG. 8A. In particular, as illustrated in FIG. 8B, the head 836 can include an engagement aperture 878 that is adapted to receive the insertion tool (not illustrated) during insertion of the compression attacher 832. The shape and depth of the engagement aperture 878 can be varied to suit the specifications of the insertion tool. In one non-exclusive embodiment, the engagement aperture 878 can be substantially hexagon-shaped and can extend into the tapered region 870 of the compression attacher 832. Alternatively, the engagement aperture 878 can have a different shape. For example, the engagement aperture 878 can be circle-shaped, oval-shaped, triangle-shaped, square-shaped, rectangle-shaped, pentagon-shaped, octagon-shaped, or some other shape.

Additionally, as illustrated in FIG. 8B, the engagement aperture 878 can include additional features, e.g., an extended and narrowed region that extends further into the tapered region 870 of the compression attacher 832, depending on the specific features of the insertion tool.

Further, FIG. 8B also illustrates certain additional dimensions of the compression attacher 832. For example, FIG. 8B illustrates a threaded length 880 and a threaded width 882 of the threaded region 860, as well as an attacher width 884, which can be defined as the maximum width of the compression attacher 832 aside from the threaded region 860.

In certain embodiments, the threaded region 860 can have a threaded length 880 of between approximately 0.30 and 0.70 inches. For example, in certain non-exclusive alternative embodiments, the threaded region 860 can have a threaded length 880 of approximately 0.30, 0.35, 0.40, 0.45, 0.50, 0.55, 0.60, 0.65 or 0.70 inches. Moreover, in some embodiments, the threaded length 880 can be between approximately 25.0 and 50.0 percent of the overall length 876 (illustrated in FIG. 8A) of the compression attacher 832. Alternatively, the threaded length 880 can be greater than 0.70 inches, less than 0.30 inches, or another value between 0.30 and 0.70 inches.

Additionally, in certain embodiments, the threaded region 860 can have a threaded width 882 of between approximately 0.125 and 0.150 inches. For example, in certain non-exclusive alternative embodiments, the threaded region 860 can have a threaded width 882 of approximately 0.125, 0.1275, 0.130, 0.1325, 0.135, 0.1375, 0.140, 0.1425, 0.145, 0.1475 or 0.150 inches. Alternatively, the threaded width 882 can be greater than 0.150 inches, less than 0.125 inches, or another value between 0.125 and 0.150 inches.

Further, in some embodiments, aside from the threaded region, the compression attacher 832 can have a maximum attacher width 884 of between approximately 0.095 and 0.120 inches. For example, in some non-exclusive alternative embodiments, the compression attacher 832 can have an attacher width 884 of approximately 0.095, 0.0975, 0.100, 0.1025, 0.105, 0.1075, 0.110, 0.1125, 0.115, 0.1175 or 0.120 inches. Alternatively, the attacher width 884 can be greater than 0.120 inches, less than 0.095 inches, or another value between 0.095 and 0.120 inches.

FIG. 8C is a top end view, i.e. looking straight on at the head 836, of the compression attacher 832 illustrated in FIG. 8A. In particular, FIG. 8C more clearly illustrates certain aspects or features of the compression attacher 832. For example, FIG. 8C illustrates the general shape of the engagement aperture 878 in the head 836 of the compression attacher 832. In this embodiment, the engagement aperture 878 is substantially hexagon-shaped in order to receive and engage a substantially hexagon-shaped insertion tool (not illustrated). Alternatively, the engagement aperture 878 can have a different shape to receive and engage a different shaped insertion tool.

Additionally, FIG. 8C further illustrates how the threads 838 of the threaded region 860 can extend out wider than the head 836 of the compression attacher 832. As noted above, the smaller head 836 is designed to have a relatively low profile to enable a low profile attacher-to-plate interface, and to allow the head 836 to pass completely through the compression aperture 22 (illustrated in FIG. 1) when and if desired.

FIG. 8D is a bottom end view, i.e. looking straight on at the tip 862, of the compression attacher 832 illustrated in FIG. 8A. In particular, FIG. 8D more clearly illustrates certain aspects or features of the tip 862 and/or the threaded region 860 of the compression attacher 832.

FIG. 8E is an enlarged view of a portion of the compression attacher 832 as indicated by circle and arrow E-E in FIG. 8B. In particular, FIG. 8E illustrates an enlarged view of the head 836, including the engagement aperture 878, and a portion of the tapered region 870 of the compression attacher 832. FIG. 8E further more clearly illustrates certain features or aspects of the compression attacher 832. For example, FIG. 8E illustrates an aperture depth 886, an aperture width 888 and a pilot angle 890 of the engagement aperture 878.

In certain non-exclusive embodiments, the aperture depth 886 can be between approximately 0.03 and 0.45 inches depending on the specific size of the insertion tool (not illustrated) with which the compression attacher 832 may be used. Alternatively, the aperture depth 886 can be greater than 0.045 inches or less than 0.03 inches. Additionally, in certain non-exclusive embodiments, the aperture width 888, e.g., diameter, can be between approximately 0.04 and 0.05 inches. Alternatively, the aperture width 888 can be greater than 0.05 inches or less than 0.04 inches.

Additionally, in some non-exclusive embodiments, the engagement aperture 878 can have a pilot angle 890 of between approximately ninety and one hundred thirty degrees. Alternatively, the engagement aperture 878 can have a pilot angle 890 of greater than one hundred thirty degrees or less than ninety degrees.

FIG. 8F is an enlarged view of a portion of the compression attacher 832 as indicated by circle and arrow F-F in FIG. 8B. In particular, FIG. 8F illustrates an enlarged view of a portion of the threaded region 860 of the compression attacher 832 that illustrates certain features or aspects of the threads 838. For example, FIG. 8F illustrates a thread spacing 892 and a thread angle 894 of the threads 838 of the threaded region 860.

In certain embodiments, the threaded region 860 can have a thread spacing 892 of between approximately 0.035 and 0.055 inches. For example, in certain non-exclusive alternative embodiments, the threaded region 860 can have a thread spacing 892 of approximately 0.035, 0.037, 0.039, 0.041, 0.043, 0.045, 0.047, 0.049, 0.051, 0.053 or 0.055 inches. Alternatively, the thread spacing 892 can be greater than 0.055 inches, less than 0.035 inches, or some other value between 0.035 and 0.055 inches.

Additionally, in certain embodiments, the thread angle 894 of the threads 838 can be between approximately 25.0 and 45.0 degrees. For example, in certain non-exclusive alternative embodiments, the thread angle 894 of the threads 838 can be approximately 25.0, 27.5, 30.0, 32.5, 35.0, 37.5, 40.0, 42.5 or 45.0 degrees. Alternatively, the thread angle 894 can be greater than 45.0 degrees, less than 25.0 degrees, or some other value between 25.0 and 45.0 degrees.

FIG. 9 is a simplified schematic illustration of a first bone part 929A, a second bone part 929B, and an embodiment of the fusion plate 10, the fixation attachers 30 and the compression attacher 32 having features of the present invention that are utilized to hold the bone parts 929A, 929B together.

As illustrated in FIG. 9, the fusion plate 10 is positioned substantially adjacent to a surface of the first bone part 929A and the second bone part 929B. Additionally, a pair of fixation attachers 30 extend through the first fixation apertures 18 positioned near the first end 12 of the fusion plate 10 and into the first bone part 929A, and a pair of fixation attachers 30 extend through the second fixation apertures 20 positioned near the second end 14 of the fusion plate 10 and into the second bone part 929B. Further, the compression attacher 32 extends through the compression aperture 22 and into or through both the first bone part 929A and the second bone part 929B, i.e. through a portion of the first bone part 929A and into the second bone part 929B. With this design, the fusion plate 10 and compression attacher 32 cooperate to enable the effective compression and/or to increase the compression of bone material at a bone interface between the bone parts 929A, 929B, i.e. to compress the bone parts 929A, 929B together.

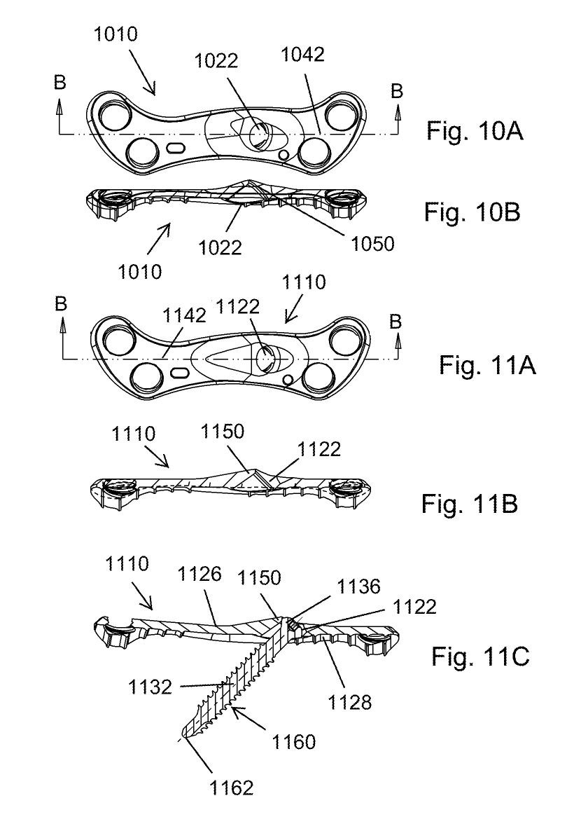

FIG. 10A is a top view of another embodiment of a fusion plate 1010 having features of the present invention. As illustrated, the fusion plate 1010 is substantially similar to the fusion plate 10 illustrated and described above. Accordingly, various features and aspects of the fusion plate 1010 will not be described in detail herein.

However, in this embodiment, the fusion plate 1010 includes a compression aperture 1022 that is somewhat different than the compression aperture 32 described in detail above. For example, in this embodiment, the compression aperture 1022 is directed at an angle relative to the longitudinal axis 1042 of the fusion plate 1010. Additionally, as shown, the compression aperture 1022 also does not include threads. With this design, the compression attacher, e.g., the compression attacher 32 and/or the compression attacher 832 illustrated herein above, can be merely extended through, and not threaded through, the compression aperture 1022.

Additionally, in this embodiment, the fusion plate 1010 is designed without a valgus angle, e.g., the valgus angle 44 illustrated in FIG. 1.

FIG. 10B is a sectional view of the fusion plate 1010 taken on line B-B in FIG. 10A. FIG. 10B illustrates that the fusion plate 1010 again includes a thickened region 1050, with the compression aperture 1022 being the non-threaded aperture that extends through the thickened region 1050 of the fusion plate 1010.

FIG. 11A is a top view of still another embodiment of a fusion plate 1110 having features of the present invention. As shown in FIG. 11A, the fusion plate 1110 is substantially similar to the fusion plate 1010 illustrated and described above in relation to FIG. 10A. For example, the compression aperture 1122 is again a non-threaded aperture for receiving the compression attacher, e.g., the compression attacher 32 and/or the compression attacher 832 illustrated herein above. However, in this embodiment, the compression aperture 1022 is directed substantially along and/or parallel to the longitudinal axis 1142.

FIG. 11B is a sectional view of the fusion plate 1110 taken on line B-B in FIG. 11A. FIG. 10B illustrates that the fusion plate 1110 again includes a thickened region 1150, with the compression aperture 1122 being the non-threaded aperture that extends through the thickened region 1150 of the fusion plate 1110.

FIG. 11C is a sectional view of the fusion plate 1110 taken on line B-B in FIG. 11A, and another embodiment of a compression attacher 1132. In particular, FIG. 11C illustrates the compression attacher 1132, e.g., a compression screw, extending through the non-threaded compression aperture 1122. In this embodiment, the compression attacher 1132 includes a threaded region 1160 that extends from the tip 1162 to near the head 1136. Additionally, the head 1136 in this embodiment is somewhat different than in the previous embodiments. For example, as illustrated, the head 1136 is rounded. However, it should be noted that even with the rounded head 1136, the head 1136 of the compression attacher 1132 does not extend above the top surface 1126 or below the bottom surface 1128 of the fusion plate 1110 due to the compression aperture 1122 extending through the thickened region 1150 of the fusion plate 1110.

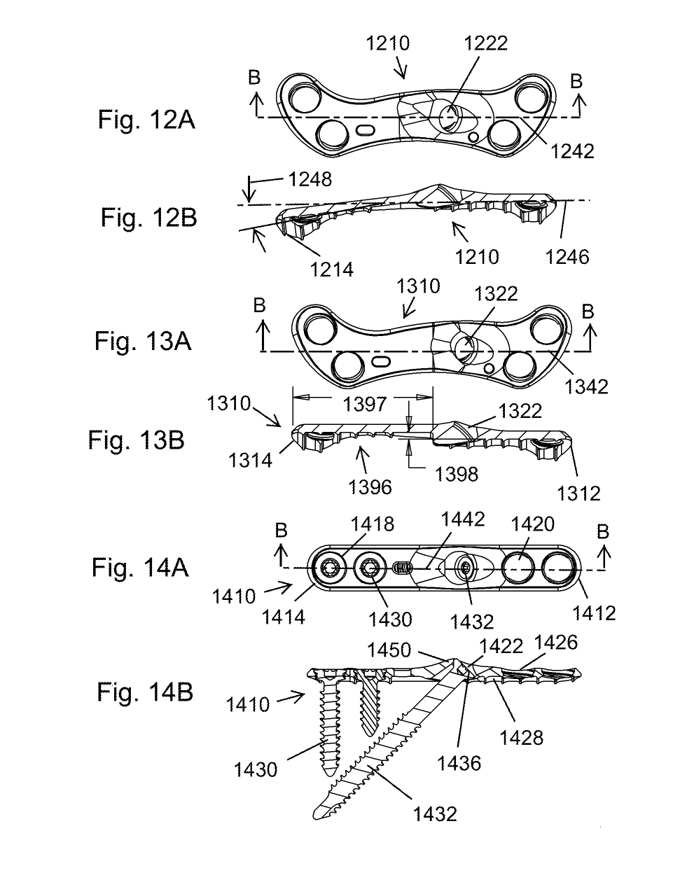

FIG. 12A is a top view of yet another embodiment of a fusion plate 1210 having features of the present invention. As illustrated, the fusion plate 1210 is substantially similar to the fusion plate 1010 illustrated and described in relation to FIG. 10A. For example, the fusion plate 1210 again includes a non-threaded, compression aperture 1222 that is directed at an angle relative to the longitudinal axis 1242 of the fusion plate 1210.

FIG. 12B is a sectional view of the fusion plate 1210 taken on line B-B in FIG. 12A. In particular, FIG. 12B illustrates certain features of the fusion plate 1210 that are different from the previous embodiments. For example, as illustrated in FIG. 12B, the second end 1214 of the fusion plate 1210 flexes in a generally downward direction at a flexion angle 1248 relative to the planar axis 1246.

FIG. 13A is a top view of still another embodiment of a fusion plate 1310 having features of the present invention. As shown in this embodiment, the fusion plate 1310 is substantially similar to the fusion plates 1010, 1210 illustrated and described in relation to FIGS. 10A and 12A, respectively. For example, the fusion plate 1310 again includes a non-threaded, compression aperture 1322 that is directed at an angle relative to the longitudinal axis 1342 of the fusion plate 1310.

FIG. 13B is a sectional view of the fusion plate 1310 taken on line B-B in FIG. 13A. As shown in FIG. 13B, the fusion plate 1310 is substantially flat. However, the fusion plate 1310 includes a small stepped region 1396 substantially adjacent to the compression aperture 1322. In particular, in this embodiment, the second end 1314 is stepped slightly in an upward direction relative to the first end 1312. Alternatively, in other embodiments, the second end 1314 can be stepped slightly in a downward direction relative to the first end 1312. The stepped region 1396 can be utilized to accommodate certain abnormalities in the bone structure in the area where the fusion plate 1310 is to be inserted. As shown in FIG. 13B, the stepped region 1396 can include a step length 1397 and a step height 1398.

In certain embodiments, the step length 1397 can be between approximately 0.65 and 1.00 inches. Alternatively, the step length 1397 can be greater than 1.00 inches or less than 0.65 inches.

Additionally, in some embodiments, the step height 1398 can be between approximately 0.02 and 0.06 inches. Alternatively, the step height 1398 can be greater than 0.06 inches or less than 0.02 inches, depending on the severity or degree of the abnormality that makes the stepped region 1396 necessary.

FIG. 14A is a top view of still yet another embodiment of a fusion plate 1410 having features of the present invention, and a plurality of fixation attachers 1430 and a compression attacher 1432 that can be used with the fusion plate 1410. In this embodiment, the fusion plate 1410 has a different shape than the previous embodiments. In particular, as illustrated in the embodiment shown in FIG. 14A, the fusion plate 1410 is substantially rectangle-shaped with rounded ends, i.e. the first end 1412 and the second end 1414 are both rounded, or semicircular, in shape.

Additionally, the fusion plate 1410 can include one or more first fixation apertures 1418 positioned near the first end 1412, and one or more second fixation apertures 1420 positioned near the second end. Further, the first fixation apertures 1418 and the second fixation apertures 1420 are substantially aligned along the longitudinal axis 1442. In this embodiment, the fusion plate 1410 includes two first fixation apertures 1418 and two second fixation apertures 1420. Alternatively, the fusion plate 1410 can include more than two or less than two first fixation apertures 1418, and/or more than two or less than two second fixation apertures 1420.

FIG. 14B is a sectional view of the fusion plate 1410, the fixation attachers 1430 and the compression attacher 1432 taken on line B-B in FIG. 14A. As shown in FIG. 14B, the fixation attachers 1430 extend in a generally vertical direction, i.e. perpendicular to the fusion plate 1410.

Additionally, the compression attacher 1432 includes a head 1436 in this embodiment is somewhat different than in the previous embodiments. For example, as illustrated, the head 1436 is flat. As with the previous embodiments, it should be noted that the head 1436 of the compression attacher 1432 again does not extend above the top surface 1426 or below the bottom surface 1428 of the fusion plate 1410 due to the compression aperture 1422 extending through the thickened region 1450 of the fusion plate 1410.

While a number of exemplary aspects and embodiments of a fusion plate 10 and a compression attacher 832 have been shown and disclosed herein above, those of skill in the art will recognize certain modifications, permutations, additions and sub-combinations thereof. It is therefore intended that the fusion plate 10 and the compression attacher 832 shall be interpreted to include all such modifications, permutations, additions and sub-combinations as are within their true spirit and scope, and no limitations are intended to the details of construction or design herein shown.

What is claimed is:

1. A fusion plate for compressing bone material at an interface between a first bone part and a second bone part, the fusion plate including a planar axis that extends along a length of the fusion plate, the fusion plate comprising:
a first end having a first thickness;
a thickened region that is spaced apart from the first end, the thickened region having a second thickness that is greater than the first thickness; and
a compression aperture that extends through the thickened region at a compression angle relative to the planar axis.

2. The fusion plate of claim 1 wherein the first end includes a top surface, and wherein the thickened region extends in a generally upward direction away from the top surface, such that the thickened region extends farther away from the bone parts than the top surface when the fusion plate is implanted substantially adjacent to the bone parts.

3. The fusion plate of claim 2 wherein the second thickness is at least approximately 1.0 millimeters greater than the first thickness.

4. The fusion plate of claim 1 wherein the compression angle is between approximately thirty and fifty-five degrees relative to the planar axis.

5. The fusion plate of claim 1 further comprising one or more plate lifters that are positioned along and extend away from a bottom surface of the fusion plate, the one or more plate lifters being adapted to engage at least one of the bone parts when the fusion plate is implanted substantially adjacent to the bone parts.

6. The fusion plate of claim 1 further comprising a first fixation aperture that extends through the fusion plate near the first end.

7. The fusion plate of claim 6 further comprising a second end that is spaced apart from the first end, and a second fixation aperture that extends through the fusion plate near the second end, and wherein the thickened region is positioned within a middle region substantially between the first end and the second end.

8. A combination comprising the fusion plate of claim 1, and a compression screw that extends through the compression aperture, the compression screw being adapted to extend into the first bone part and the second bone part when the fusion plate is implanted substantially adjacent to the bone parts.

9. The combination of claim 8 wherein the compression screw includes (i) a threaded region including a plurality of threads having a threaded width, and (ii) an attacher width, and wherein the threaded width is greater than the attacher width.

10. A fusion plate for compressing bone material at an interface between a first bone part and a second bone part, the fusion plate including a planar axis that extends along a length of the fusion plate, the fusion plate comprising:
a top surface;
an opposed bottom surface; and
one or more plate lifters that are positioned along and extend away from the bottom surface of the fusion plate, the one or more plate lifters adapted to engage at least one of the bone parts when the fusion plate is implanted substantially adjacent to the bone parts, the one or more plate lifters having a buttress design.

11. The fusion plate of claim 10 further comprising a compression aperture that extends through the top surface and the bottom surface at a compression angle relative to the planar axis.

12. The fusion plate of claim 11 wherein the compression angle is between approximately thirty and fifty-five degrees relative to the planar axis.

13. The fusion plate of claim 11 further comprising a first end having a first thickness; and a thickened region that is spaced apart from the first end, the thickened region having a second thickness that is greater than the first thickness; and wherein the compression aperture extends through the thickened region.

14. The fusion plate of claim 13 wherein the top surface extends at least from the first end to the thickened region, and wherein the top surface at the thickened region extends farther away from the bone parts than the top surface at the first end when the fusion plate is implanted substantially adjacent to the bone parts.

15. The fusion plate of claim 13 wherein the second thickness is at least approximately 1.0 millimeters greater than the first thickness.

16. The fusion plate of claim 13 further comprising (i) a second end that is spaced apart from the first end, the thickened region being positioned within a middle region substantially between the first end and the second end, (ii) a first fixation aperture that extends through the fusion plate near the first end, and (iii) a second fixation aperture that extends through the fusion plate near the second end.

17. A combination comprising the fusion plate of claim 11, and a compression screw that extends through the compression aperture, the compression screw being adapted to extend into the first bone part and the second bone part when the fusion plate is implanted substantially adjacent to the bone parts.

18. A combination for compressing bone material at an interface between a first bone part and a second bone part, combination comprising:
a fusion plate including (i) a first end having a top surface and a first thickness, (ii) an opposed second end that is spaced apart from the first end, (iii) a thickened region that is positioned within a middle region substantially between the first end and the second end, the thickened region extending in a generally upward direction away from the top surface, such that the thickened region extends farther away from the bone parts than the top surface when the combination is implanted substantially adjacent to the bone parts, the thickened region having a second thickness that at least approximately 1.0 millimeters greater than the first thickness, and (iv) a compression aperture that extends through the thickened region at a compression angle of between approximately thirty and fifty-five degrees relative to the planar axis; and
a compression screw that extends through the compression aperture, the compression screw being adapted to extend into the first bone part and the second bone part when the combination is implanted substantially adjacent to the bone parts.

19. The combination of claim 18 wherein the fusion plate further includes one or more plate lifters that are positioned along and extend away from a bottom surface of the fusion plate, the one or more plate lifters being adapted to engage at least one of the bone parts when the fusion plate is implanted substantially adjacent to the bone parts.

20. The combination of claim 18 wherein the compression screw includes (i) a threaded region including a plurality of threads having a threaded width, and (ii) a screw width, and wherein the threaded width is greater than the screw width.
